(19) United States
(12) Patent Application Publication
(10) Pub. No.: US 2008/0132465 A1
(43) Pub. Date: Jun. 5, 2008

(54) APPARATUS AND METHOD FOR ISOLATING IRON COMPONENTS FROM SERUM

(76) Inventors: Vincent Windisch, Collegeville, PA (US); Pui-Ho Yuen, Princeton Junction, NJ (US); Jing Jun Huang, Monmouth, NJ (US); Jingzhen Xu, Delran, NJ (US)

Correspondence Address: BAXTER HEALTHCARE CORPORATION, 1 BAXTER PARKWAY, DF2-2E, DEERFIELD, IL, 60015, US

(21) Appl. No.: 11/567,029

(22) Filed: Dec. 5, 2006

(51) Int. Cl. A61K 31/7016; C07H 23/00; B01D 11/00; G01N 21/01; G01N 33/20

(52) U.S. Cl. 514/53; 536/121; 210/511; 422/82.05; 73/866

(57) ABSTRACT

A method and apparatus for isolating iron components in serum is provided. The method includes passing a serum sample through a separation column having a solid phase and retaining with the solid phase an iron component, such as an iron carbohydrate, in the column. Elution of the solid phase may retrieve an eluate containing the iron component. The amount of iron component present in the eluate may then be measured. The present method and apparatus advantageously permit the direct measurement of the iron component present in the serum.

### BACKGROUND

Iron is essential for the synthesis of hemoglobin and the maintenance of oxygen transport as well as for the function and formation of other physiologically important heme and non-heme compounds. Iron deficiency anemia, a blood disorder, is commonly treated with the oral administration of iron salts, such as ferrous sulfate, ferrous gluconate, ferrous fumarate, and ferrous orotate. However, many patients with severe iron depletion or for whom oral administration of iron is ineffective, such as non-dialysis dependent chronic kidney disease patients, hemodialysis patients, and peritoneal dialysis patients, often require intravenous or parenteral administration of an iron carbohydrate to maintain sufficient iron stores within the body. Iron carbohydrates, and iron dextran in particular, often cause adverse side effects such as anaphylaxis, hypersensitivity, skin staining at the injection site, fever, and severe allergic reaction when administered to patients.

One type of iron carbohydrate, namely, iron saccharidic complex, which includes sodium ferric gluconate and ferric hydroxide-sucrose complexes (or iron sucrose), has been developed to reduce the adverse side effects of iron dextran. Iron saccharidic complexes are characterized by a ferric iron that is present in a spatial complex with a saccharide or a disaccharide such as sucrose or a sucrose derivative. In the case of iron sucrose, the sucrose component is composed of disaccharides and typically lacks polysaccharide derivatives.

Accurate quantitative detection of serum iron components in the presence of iron carbohydrates has proven problematic. For example, iron sucrose present in serum tends to interfere with conventional techniques for transferrin-bound iron (TBI) detection that utilize colorimetry and/or spectrophotometry. Such techniques typically require the reduction of ferric iron to ferrous iron and chelation of the ferrous iron to form a colored complex. These procedures, however, fail to accurately distinguish between iron originating from the iron carbohydrate, such as an iron saccharidic complex, and iron originating from TBI. In particular, conventional detection techniques requiring ferric reduction not only release iron from TBI but also release iron from the iron carbohydrate. This results in an overestimation of TBI present in serum. An inaccurate measurement of TBI may cause misdiagnosis—a false detection of TBI over-saturation indicates the existence of free iron, a condition that may cause acute toxicity.

A need therefore exists for a method and apparatus for the isolation of iron components in blood serum that contains an iron carbohydrate such as an iron saccharidic complex. A need further exists for a method and apparatus for isolating and directly measuring the amount of transferrin-bound iron and/or the amount of iron carbohydrate present in blood serum from patients administered with an iron carbohydrate.

### SUMMARY

The present method advantageously eliminates the interference of iron carbohydrate, and iron saccharidic complexes in particular, in TBI detection for serum containing an iron carbohydrate. This permits an accurate measurement of TBI when an iron carbohydrate such as an iron saccharidic complex is also present in blood serum. Moreover, the present disclosure concomitantly provides an accurate method for determining and measuring the concentration of an iron carbohydrate in serum. An iron carbohydrate, such as an iron saccharidic complex, may be isolated from the serum permitting the direct measurement of iron saccharidic complex in serum. Direct measurement of the iron carbohydrate in serum further provides for an accurate and simple procedure to determine the bioavailability of an iron carbohydrate.

In an embodiment, a method for isolating an iron component from serum is provided. The method includes introducing a serum sample into a separation column having a solid phase. The solid phase retains an iron component present in the serum sample. The solid phase may include a substrate with a functional group such as a cyano, a diol, an amino, an alkyl amino, a dimethylamino, and a primary/secondary amine group. The method further includes removing the iron component from the solid phase. The iron component may be any iron composition administered to treat iron deficiency anemia as is commonly known in the art. For example, the iron component may be an iron carbohydrate or an iron saccharidic complex. In an embodiment, the iron component may be iron sucrose.

In an embodiment, the iron component may be removed from the solid phase by passing an eluant through the column and collecting an eluate from the column. Once the eluate containing the iron component is collected, the amount of iron component in the eluate may be measured. The eluant may be passed through the column repeatedly as desired to ensure substantially all, or all, of the iron component is retrieved from the solid phase.

In an embodiment, the method may include eluting a buffer solution through the column prior to the removing. The buffer solution may wash any free iron or other residual iron from the solid phase.

In an embodiment, the removal of the iron component may include lowering the pH of the solid phase. This may be accomplished by passing an acid eluant through the column. In an embodiment, the acid eluant may have a pH less than about 6.0.

In an embodiment, the removal of the iron component may include raising the pH of the solid phase. This may be accomplished by passing a base eluant through the column. In an embodiment, the base eluant may have a pH greater than about 8.0.

In an embodiment, the method may include collecting a serum eluate from the column. As the solid phase may retain all, or substantially all, of the iron component in the serum, the serum eluate may contain transferrin-bound iron, or transferrin-bound iron that is free of the iron component. The amount of transferrin-bound iron present in the serum eluate may be measured. In an embodiment, the serum eluate may be collected from the column before the iron component is removed from the solid phase.

In an embodiment, an apparatus for isolating an iron component from serum is provided. The apparatus includes a separation column having a solid phase for retaining the iron component from a serum sample passed therethrough. The solid phase may include a substrate material with a functional group such as a cyano, a diol, an amino, an alkyl amino, a dimethylamino, and a primary/secondary amine group. The apparatus may further include an eluant for removing the iron component from the solid phase, and a container for receiving an eluate from the column.

The iron component may be any iron composition administered to treat iron deficiency anemia as is commonly known in the art. For example, the iron component may be an iron carbohydrate or an iron saccharidic complex. In an embodiment, the iron component may be iron sucrose.

In an embodiment, the apparatus may include a buffer solution. The buffer solution may be used to wash the column and solid phase at any stage as desired. The buffer solution may be passed through the column either before or after the serum is introduced into the column. Similarly, the buffer solution may be passed through the column when the iron component is retained by the solid phase. The buffer solution may remove any spurious TBI and/or free iron bound to the solid phase when eluted through the column. In an embodiment the buffer solution may be a phosphate buffered saline solution.

In an embodiment, the apparatus may include an acid eluant. Passage of the acid eluant through the column may remove the iron component from the solid phase. In an embodiment, the acid eluant may have a pH less than about 6.0.

In an embodiment, the eluant may be a base eluant. Passage of the base eluant through the column may remove the iron component from the solid phase. In an embodiment, the base eluant may have a pH greater than about 8.0.

In an embodiment, the apparatus may include a measurement device for determining the amount of iron component present in the eluate. The measurement device may also be used to determine the amount of TBI present in the serum eluate. The measurement device may be a calorimeter, a spectrophotometer, an inductively coupled plasma—atomic emission spectrometer, an inductively coupled plasma—mass spectrometer, an atomic absorption spectrometer, and combinations thereof.

In an embodiment, a method for determining the bioavailability of an iron sucrose composition is provided. The method includes obtaining a serum sample containing iron sucrose and isolating the iron sucrose from the serum sample. Upon isolation from the serum sample, the amount of iron sucrose present in the sample may be measured.

In an embodiment, the iron sucrose may be isolated from the serum by introducing the serum sample into a separation column having a solid phase and retaining with the solid phase the iron sucrose. The solid phase may be include a functional group as previously discussed herein.

In an embodiment, the method may include passing an eluant through the column to remove the iron sucrose from the solid phase. An eluate containing the iron sucrose may then be collected from the column. The eluant may be an acid eluant. Alternatively, the eluant may be a base eluant.

In an embodiment, the method may include administering an iron sucrose composition to a subject. One or more serum samples may be obtained from the subject at one or more predetermined time intervals after the iron sucrose administration. In a further embodiment, serum samples may be obtained after iron sucrose administration at a time interval of 0 minutes, 1 minute, 5 minutes, 10, minutes, 15 minutes, 30 minutes, 45 minutes, 1 hour, 2 hours, 3 hours, 4 hours, 6 hours, 8 hours, 12 hours and 24 hours and combinations thereof.

In an embodiment, the method may include obtaining a plurality of serum samples at a plurality of time intervals and measuring the amount of iron sucrose present in each sample. The method may include comparing the amount of iron sucrose present in each sample with a second serum sample containing a second iron sucrose composition having a known bioavailability. By comparing the amount of iron sucrose present in each sample with a second serum sample having a known bioavailability, the bioavailability of the iron sucrose composition may be determined.

In a further embodiment, the method may include obtaining a plurality of serum samples from one or more subjects administered with the iron sucrose composition. Serum samples may be obtained from the subjects, the amount of iron sucrose in each sample being measured as disclosed herein. In an embodiment, the plurality of serum samples obtained from different time intervals may be used to prepare a pharmacokinetic analysis for iron sucrose based on the iron sucrose concentration value of each sample. This analysis may be based on the reduction of iron sucrose concentration in the serum over time. The pharmacokinetic analysis prepared by way of the direct measurement of iron sucrose concentration in serum may subsequently be compared to the pharmacokinetic properties of a second iron sucrose composition having a known bioavailability. In this sense, the bioequivalence of an iron sucrose composition may be determined by way of direct measurement of iron sucrose concentration in the serum.

It is an advantage of the present disclosure to provide a method that isolates an iron component, such as an iron carbohydrate, from serum. It is a further advantage of the present disclosure to isolate an iron component from a serum sample that also contains transferrin-bound iron.

It is an advantage of the present disclosure to directly measure the amount of an iron carbohydrate present in serum. Direct measurement provides a more accurate determination of the amount of iron carbohydrate present in serum compared to protocols that indirectly determine the quantity of iron carbohydrate in serum.

It is an advantage of the present disclosure to isolate transferrin-bound iron in serum. It is a further advantage to isolate transferrin-bound iron from serum that contains other iron components.

It is an advantage of the present disclosure to isolate an iron component, such as an iron carbohydrate, from transferrin-bound iron in serum.

Additional features and advantages are described herein, and will be apparent from, the following Detailed Description.

### BRIEF DESCRIPTION OF THE FIGURES

FIG. 1 is a graph depicting a curve based on iron sucrose concentrations in human serum in accordance with an embodiment of the present disclosure.

### DETAILED DESCRIPTION

In an embodiment, the present disclosure provides a method for isolating an iron component from serum. The method may include introducing or otherwise passing a serum sample into a separation column having a solid phase. The separation column may include a solid phase that may be adapted to retain the iron component. This, the solid phase may retain any iron component that may be present in the serum sample. Once retained by the solid phase, the method may further include removing the iron component from the solid phase.

The iron component may be any iron carbohydrate administered for the treatment of iron deficiency anemia as is commonly known in the art. Nonlimiting examples of suitable iron components include iron carbohydrates administered intravenously, iron disaccharide complexes, iron dextran, ferric gluconate, and iron sucrose. In an embodiment, the iron component may be iron sucrose.

Iron sucrose, an iron saccharidic complex, is a polynuclear (i.e., containing multiple linked iron atoms) composition composed of colloidal ferric, iron (III), hydroxide particles in complex with sucrose. Iron sucrose is typically prepared as an aqueous solution and has a molecular weight of approximately 34,000-60,000 Daltons. with the formula as provided below:

[Na₂Fe₅O₈(OH).3(H₂O)]ₙ.m(C₁₂H₂₂O₁₁)

where n is the degree of iron polymerization and m is the number of sucrose molecules associated with the iron(III)-hydroxide. Not wishing to be bound to any particular theory, it is believed that the molecular structure of iron sucrose includes an iron oxyhydroxide (β-FeOOH) core, the core bonding with the sucrose by way of a non-covalent intermolecular force, such as the attraction of partial positive charges of iron atoms to the negative dipole moments of the sucrose hydroxyl groups.

Iron sucrose is typically administered intravenously to replenish depleted blood iron stores in anemic patients, and dialysis patients in particular. An iron sucrose composition typically includes about 100 mg elemental iron in 5 ml of a water solution (20 mg free iron/ml), and 30% sucrose w/v (300 mg/ml). An iron sucrose composition typically has a pH from about 10.5 to about 11.1, and is negatively charged at alkaline pH. In an embodiment, the iron sucrose composition may be diluted in a 0.9% by weight sodium chloride solution having a pH of about 10.0. Comparatively, iron dextran typically constitutes a larger molecule (approximately 165 kD to 265 kD) than iron sucrose and is typically administered as 100 mg iron in 2 ml water solution. The pH of iron dextran may range from about 5.2 to about 6.5. Iron dextran includes polysaccharides whereas the disaccharides of iron sucrose are not polymerized.

In an embodiment, the separation column may be any column that utilizes a solid phase and a liquid phase to isolate an analyte from a solution or a liquid as is commonly known in the art. Nonlimiting examples of suitable separation columns include ion exchange columns and solid phase extraction (SPE) columns. The column may have a volume from about 1 ml to about 60 ml or any volume therebetween. In an embodiment, the separation column may be an SPE column. Solid-phase extraction is an extraction method that typically uses inert particles about 3-300 μm in diameter as the solid phase. The particles may be regularly shaped, irregularly shaped, or a combination thereof. Not wishing to be bound to any particular theory, separation in the column occurs as the eluant travels through the solid phase, with analytes separating from the eluant due to the differences in the partitioning between the liquid phase and the solid phase. Analyte separation or retention may include absorption, adsorption, ion exchange, hydrogen bonding, dipole/dipole attraction, dipole/induced dipole attraction, or similar intramolecular attractions between analyte molecules and the solid phase.

In an embodiment, the solid phase may include a substrate, such as silica particles, with a polar functional group attached thereto. Alternatively, the solid phase may be a fine particulate metal, such as alumina, for example. In a further embodiment, the substrate particles may be coated with a polar functional group. Nonlimiting examples of suitable functional groups include cyano (—C₂H₄CN), diol (—C₃H₆OCH₂CHOHCH₂OH), amino (—NH₂), alkyl amino (—C₃H₆NH₂), dimethylamino (—C₃H₆N(CH₃)₂, or primary/secondary amine (—CH₂CH₂NHCH₂CH₂NH₂). In a further embodiment, the functional group may be an amino group. In yet a further embodiment, an alkyl amino group may be adhered to the silica particles, the alkyl amino group represented by the structure below:

Si—CₓH₂NH₂

wherein x equals the number of carbon atoms in the alkyl group, and x may be from 1 to about 10. In yet a further embodiment, silica particles coated with a polar functional group may form a solid phase bed in the separation column, the solid phase bed having a mass from about 30 mg to about 20 g, or any mass therebetween.

In an embodiment, the functional group may be an alkyl amino group. The alkyl amino group may form a polar sorbent and may utilize both hydrogen bonding and/or ion or anion exchange. In a further embodiment, the pKa of the alkyl amino group may be about 10.0. Consequently, at a pH below about 10, the alkyl amino group may be positively charged whereas a pH equal to or above about 10 results in a neutral alkyl amino group.

The solid phase may effectively retain all, or substantially all, of the iron component that may be present in the serum sample. In an embodiment, passage of the serum through the column may yield a serum eluate containing no, or substantially no, iron component. For example, in the event the iron component is iron sucrose, the serum eluate may be substantially free, or free, of iron sucrose.

In an embodiment, the method may include collecting a serum eluate from the column. The serum eluate may include transferrin-bound iron (TBI). The method may further entail measuring the amount of TBI present in the serum eluate. The amount or concentration of TBI present in the serum eluate may be determined by a measurement device capable of providing quantitative chemical analysis of the serum eluate as is commonly known in the art. Nonlimiting examples of suitable measurement devices include a colorimeter, a spectrophotometer, an inductively coupled plasma-atomic emission spectrometer (ICP-AES), an inductively coupled plasma-mass spectrometer (ICP-MS), an atomic absorption spectrometer, and combinations thereof. As the solid phase retains all (or substantially all) of the iron component present in the serum, the present method advantageously provides a simple, quick, accurate, and effective process in which to precisely measure TBI concentration in serum for subjects administered with an iron carbohydrate without interference from the iron carbohydrate.

In an embodiment, the method may further include removing the iron component retained by the solid phase by introducing or otherwise passing an eluant into and through the separation column and collecting an eluate from the column. The eluate may contain the iron component present in the serum sample. In an embodiment, the removal of the iron component from the solid phase may include lowering the pH of the solid phase by passing or otherwise eluting an acid eluant through the separation column. In an embodiment, the acid eluant may have a pH less than about 6.0, or from about 5.5 to about 3.0, or any value therebetween. Not wishing to be bound to any particular theory, it is believed that the pH of the column during serum sample passage creates an environment wherein the solid phase of the separation column may have a positive charge or a positive attraction force which attracts the iron component having a negative charge or a negative attraction force. The negative charge of the iron component may be due to the provision of an overall negative charge or negative attraction force by the hydroxyl groups of the iron oxyhydroxide at neutral or slightly alkaline pH. In an acidic environment, the pH of the column may neutralize the negative charge of the hydroxyl groups of the iron component. This makes the iron component more positive in charge, eliminating the attractive forces between the solid phase and the iron component, and thereby permitting the iron component to elute through the column with the acid eluant.

In an embodiment, the removal of the iron component from the solid phase may include raising the pH of the solid phase by passing or otherwise eluting a base eluant through the separation column. The base eluant may have a pH greater than about 8.0 or greater than about 10.0, or from about 10.0 to about 12.0, or any value therebetween. In a further embodiment, the base eluant may have a pH equal to or greater than the pKa of the functional group for the solid phase. Not wishing to be bound by any particular theory, it is believed that introduction of the base eluant into the separation column alters the pH within the column. The base eluant may alter the pH of the solid phase and neutralize the positive charge of the solid phase that is present during serum sample elution. This may remove the attractive forces between the solid phase and the iron component, thereby eliminating the retention of the iron component by the solid phase. Consequently, the iron component elutes through the solid phase with the base eluant, permitting the iron component to be collected. In a further embodiment, the removal and collection of the iron component eluate may be performed at ambient conditions.

In an embodiment, the column may be washed with a buffer solution to wash or otherwise remove any spurious TBI or exogenous iron that may have been retained by the solid phase. This may be performed prior to introduction of the acid eluant or the base eluant into the column. The buffer solution may be any aqueous solution capable of washing free iron/TBI from the column. In a further embodiment, the buffer solution may be a phosphate buffered saline solution having a pH of about 7.4. The phosphate buffered saline solution may have a concentration of 0.01 M.

In a further embodiment, removal of the iron component from the separation column may be performed rapidly, as within less than about 10 seconds or less than about 5 seconds, for example. This may be accomplished by applying a positive pressure to the eluant as it is introduced into and passes through the column. In an embodiment, a syringe adapter may be placed on the top of the column. Application of a negative pressure to the column (i.e., a suction force at the bottom end of the column) to hasten elution is also contemplated. A syringe with the acid or base eluant may then be used to inject, under pressure, the acid or base eluant into and through the solid phase in a rapid manner. In an embodiment, elution may occur in about five seconds or less. This step may be repeated as desired to ensure all, or substantially all, the iron component is removed from the solid phase.

Once the eluate containing iron component is retrieved from the column, the amount of iron component in the eluate (i.e., the iron component concentration of the original serum sample) may be measured by any measurement device capable of quantitatively determining the amount of iron component present in the eluate. The measurement device may be any of the aforementioned measurement devices or any suitable combination thereof.

The present method advantageously provides a procedure in which serum iron components and TBI may be isolated and measured directly. This overcomes a shortcoming in conventional serum iron component detection methodology whereby the iron component concentration in serum is determined indirectly. For example, iron sucrose concentration in serum is conventionally determined by first determining the amount of total iron present in serum and subtracting the amount of TBI from the total iron value. This yields an indirect measurement of the iron sucrose concentration in the serum. By directly measuring the amount of iron sucrose in the serum, the present method provides for a simple, cost-effective, more accurate, more reliable, and more precise measurement of iron component concentration in serum than afforded by conventional indirect measurement techniques. It is understood that this direct measurement approach may be applied to other iron carbohydrates.

In an embodiment, the present disclosure provides an apparatus or kit for isolating an iron component from serum. The apparatus may include a separation column having a solid phase for retaining the iron component from a serum sample passed through the column. The separation column may be any column and may include any solid phase as discussed herein. In an embodiment, the iron component may be any iron component as discussed herein. In a further embodiment, the iron component may be iron sucrose.

The apparatus may include an eluant for removing the iron component from the solid phase and a container for receiving an eluate from the column. The eluant may be an acid eluant or a base eluant as discussed herein. In a further embodiment, the apparatus may include a buffer solution for washing the solid phase. The buffer solution may be any buffer solution discussed herein and may be introduced into the column before introduction of the serum sample, or after introduction of the serum sample into the column.

In an embodiment, the apparatus may also include a column-to-syringe adapter with eluants being introduced into the column by way of a syringe or a pipette (e.g., a digital pipette) as is commonly known in the art. The apparatus may further include a quantity of purified water for initially washing the solid phase prior to serum sample introduction into the column.

In an embodiment, the apparatus may include a measurement device for determining the amount of iron component present in the eluate. The measurement device may be any measurement device as previously discussed herein. The measurement device may also be used to detect the amount of TBI present in the serum eluate.

In an embodiment, a method for determining the bioavailability of an iron sucrose composition is provided. The method may include obtaining a serum sample containing iron sucrose, isolating the iron sucrose from the serum sample, and measuring the amount of iron sucrose present in the sample. The iron sucrose may be isolated by introducing the serum sample into a separation column having a solid phase and retaining with the solid phase the iron sucrose. An eluant may be passed through the column to remove the iron sucrose from the solid phase. The eluate containing the iron sucrose may be collected in a suitable container.

Bioavailability may be defined as the rate and extent to which an active ingredient or active moiety is absorbed from a drug product and becomes available at the site of action. Bioequivalence may be defined as the absence of a significant difference in the rate and extent to which the active ingredient or active moiety in pharmaceutical equivalents or pharmaceutical alternatives becomes available at the site of drug action when administered at the same molar dose under similar conditions.

In an embodiment, the method may include administering an iron sucrose composition to a subject and subsequently obtaining the serum sample from the subject at one or more pre-determined time intervals after administration of the iron sucrose. The subject may be a human or a mammal as desired. Retrieval of serum samples from the subject may occur at time intervals over a predetermined time period after iron sucrose administration. In other words, the serum samples may be obtained from the subject after iron sucrose administration at designated times. In an embodiment, serum samples may be obtained at the following post-administration time intervals: 0 minutes, 1 minute, 5 minutes, 10, minutes, 15 minutes, 30 minutes, 45 minutes, 1 hour, 2 hours, 3 hours, 4 hours, 6 hours, 8 hours, 12 hours, 24 hours, 36 hours, 48 hours and any combination thereof or any time increment therein. In a further embodiment, one or more serum samples may be obtained from the subject prior to iron sucrose administration. It is understood that serum samples may be from one subject or a plurality of different subjects, each subject administered with a predetermined amount of iron sucrose.

In an embodiment, the method may include obtaining a plurality of serum samples at a plurality of different time intervals and measuring the amount of iron sucrose present in each sample. Based on the iron sucrose concentration values determined for each sample, the amount of iron sucrose absorbed into the blood serum and the absorption rate of the iron sucrose by the subject may consequently be calculated. One of ordinary skill in the art will appreciate that after administration of the iron sucrose into the subject, the iron sucrose concentration in the collected serum samples will typically decrease over time. Thus, the elimination rate of iron sucrose from the serum may be correlated to the iron absorption rate by the body. In a further embodiment, the method may include isolating transferrin bound iron from each sample and measuring the concentration of transferring bound iron in each sample. Thus, the values for iron sucrose concentration and transferrin bound iron concentration may be used to determine or calculate the absorption rate of iron into the blood and/or iron absorption rate into the systemic circulation of the subject.

In an embodiment, the method may further include preparing a pharmacokinetic analysis for iron sucrose based on the iron sucrose concentration value obtained for each sample. One of ordinary skill in the art will recognize that the reduction of iron sucrose concentration in the samples over time may serve as an indication of the rate of metabolism of iron sucrose by the subject. Consequently, values obtained from the direct measurement of iron sucrose concentration alone or with the direct measurement of TBI concentration from serum samples taken periodically post administration may be used to prepare pharmacokinetic metrics such as iron metabolism rate, iron elimination rate, residence time, etc., for the generation of a comprehensive pharmacokinetic analysis for iron sucrose. In a further embodiment, the method may include comparing the pharmacokinetic analysis based on the iron sucrose concentration values, alone or in connection with TBI concentration values, to the pharmacokinetic properties of an iron sucrose composition having a known bioavailability (i.e., an iron sucrose composition approved for use by the U.S. Food and Drug Administration or other foreign governmental regulatory agency equivalent). It is understood that the aforementioned techniques may be applied to determine the bioavailability, the bioequivalence, and the pharmacokinetic properties for other iron carbohydrates.

In an embodiment, the method may include isolating the iron sucrose using a separation column as discussed above. In particular, the bioavailability determination method may include passing the serum sample through a separation column and retaining the iron sucrose with a solid phase of the separation column. A serum eluate may be retrieved from which TBI (in the absence of iron sucrose) concentration may be measured as commonly known in the art. The method may further include, removing with an eluant the iron sucrose retained by the solid phase, and collecting an iron sucrose eluate from the column as previously discussed herein. The eluate may be an acid eluate or a base eluate as previously discussed. The iron sucrose concentration for each iron sucrose eluate obtained may be measured by any suitable quantitative analysis procedure as previously set forth herein.

The present method advantageously provides a method for determining bioavailability/bioequivalence of an iron sucrose composition by way of direct isolation of iron sucrose and direct measurement of iron sucrose concentration in the subject's serum. Isolation of the iron sucrose also permits the direct measurement of TBI concentration in serum. Known are bioequivalence studies for iron sucrose complex that determine the concentration of iron sucrose complex in serum by an indirect approach. In particular, the iron sucrose concentration in serum is calculated by initially determining the total iron concentration in the serum and subtracting from this value the concentration of transferrin bound iron. The present bioavailability determination method, on the other hand, advantageously utilizes a direct measurement of the iron sucrose complex. Direct measurement of iron sucrose concentration for pharmacokinetic analysis is advantageous as it results in a more precise and accurate portrayal of the characteristics and properties of iron sucrose in blood, and iron metabolism rate in the blood in particular. The present method provides the direct quantization of iron sucrose and TBI in serum to yield a more accurate method of determining the bioavailability of an iron sucrose composition.

By way of example and not limitation, examples will now be given.

### EXAMPLES

### Example 1

Materials:

Solid Phase Extraction Device (SPE) (e.g., Phenomenex, Strata NH₂, 50 mg/6 mL)

Purified water

Procedure:
Step 1. Conditioning: Apply and elute approximately 4 mL of purified water into the tube.
Step 2. Sample Loading: Transfer 1.5 mL of serum sample into the SPE tube.
Step 3. Sample Elution: Allow the serum to elute from the tube. Discard the first 20 drops (approximately 750 uL) of serum to waste and collect the remaining serum sample in an appropriate container for analysis.

### Example 2

Materials:
Solid Phase Extraction Device (e.g., Phenomenex, Strata NH₂),
Phosphate Buffered Saline (PBS)
Purified water

Procedure:
Step 1. Conditioning: Apply and elute approximately 4 mL of purified water into the tube.
Step 2. Sample Loading: Quantitatively pipette and transfer 1.5 mL of serum sample and elute from the SPE tube.
Step 3. Wash: Elute 3 mL of PBS to waste to remove remaining TBI and serum.
Step 6. Elute Iron Sucrose Composition: Apply 5.0 mL of 0.1 N NaOH. Elute the 0.1 NaOH into a suitably volumetric flask. Elution of the composition is performed very rapidly by applying excess pressure onto the tube.

Quantitation of Iron Sucrose in Human Serum

I. Linearity

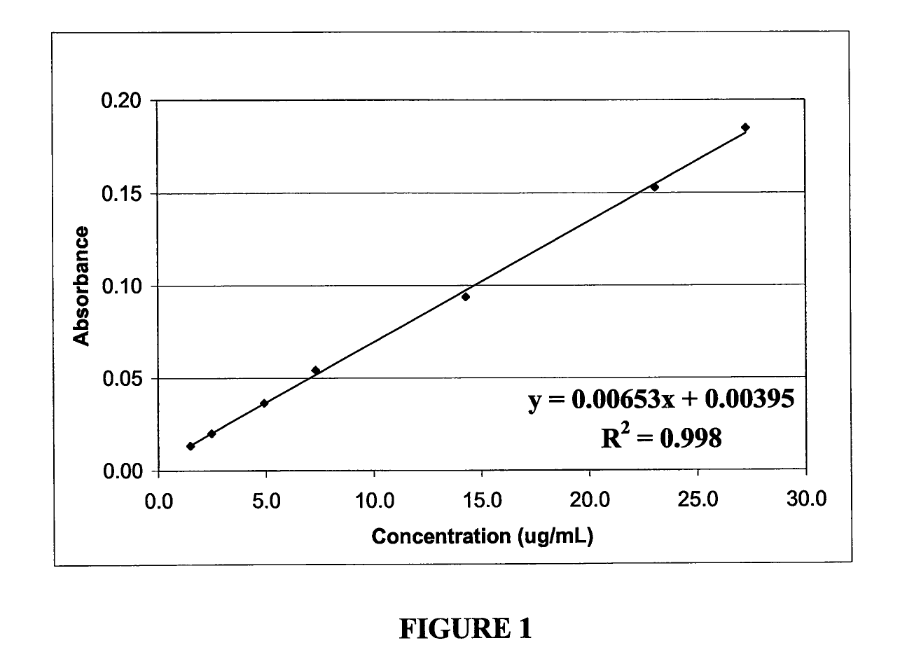

A standard curve was constructed from iron sucrose injection in human serum at target concentrations of 1.5, 2.5, 5.0, 7.5, 15.0, 25.0, 30.0 μg/mL of iron. The method was linear with a correlation coefficient, r²=0.998, as shown in the plot set forth in FIG. 1.

II. Accuracy and Precision

Samples were prepared at target concentrations of 25.0 and 7.5 μg/mL iron sucrose (as elemental iron) in human serum. Five replicate assays were performed on each sample to determine the accuracy and precision of the method as shown in the table below. The accuracy was calculated as a percent of actual and the precision is the percent relative standard deviation (or coefficient of variation). The accuracy and precision values are within ±15%, which is consistent with the FDA guidance document, Bioanalytical Method Validation.

| Target Concentration | Accuracy | Precision |
|---|---|---|
| 25.0 μg/mL | 103.1% | 3.3% |
| 7.5 μg/mL | 108.5% | 4.2% |

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method for isolating an iron component from serum comprising:
introducing a serum sample into a separation column having a solid phase;
retaining with the solid phase an iron component present in the serum sample; and
removing the iron component from the solid phase.

2. The method of claim 1 wherein the iron component is iron sucrose.

3. The method of claim 1 wherein the removing further comprises passing an eluant through the column and collecting an eluate.

4. The method of claim 3 further comprising measuring the amount of iron component in the eluate.

5. The method of claim 3 further comprising repeating the passing.

6. The method of claim 1 further comprising collecting, before the removing, a serum eluate from the column, the serum eluate containing transferrin-bound iron.

7. The method of claim 6 further comprising measuring the amount of transferrin-bound iron present in the serum eluate.

8. The method of claim 1 wherein the solid phase further comprises a substrate with a functional group selected from the group consisting of cyano, diol, amino, alkyl amino, dimethylamino, and primary/secondary amine.

9. The method of claim 1 further comprising eluting a buffer solution through the column prior to the removing.

10. The method of claim 1 wherein the removing further comprises lowering the pH of the solid phase with an acid eluant having a pH less than about 6.0

11. The method of claim 1 wherein the removing further comprises raising the pH of the solid phase with a base eluant having a pH greater than about 8.0.

12. An apparatus for isolating an iron component from serum comprising:
a separation column having a solid phase for retaining the iron component from a serum sample passed therethrough;
an eluant for removing the iron component from the solid phase; and
a container for receiving an eluate from the column.

13. The apparatus of claim 12 wherein the solid phase further comprises a substrate material with a functional group selected from the group consisting of cyano, diol, amino, alkyl amino, dimethylamino, and primary/secondary amine.

14. The apparatus of claim 12 wherein the iron component is iron sucrose.

15. The apparatus of claim 12 further comprising a buffer solution for washing the solid phase.

16. The apparatus of claim 12 wherein the eluant is an acid eluant having a pH less than about 6.0.

17. The apparatus of claim 12 wherein the eluant is a base eluant having a pH greater than about 8.0.

18. The apparatus of claim 12 wherein the apparatus further comprises a measurement device for determining the amount of iron component in the eluate, the measurement device selected from the group consisting of a colorimeter, a spectrophotometer, an inductively coupled plasma-atomic emission spectrometer, an inductively coupled plasma-mass spectrometer, an atomic absorption spectrometer, and combinations thereof.

19. A method for determining the bioavailability of an iron sucrose composition, the method comprising:
obtaining a serum sample containing iron sucrose;
isolating the iron sucrose from the serum sample; and
measuring the amount of iron sucrose present in the sample.

20. The method of claim 19 wherein the isolating further comprises introducing the serum sample into a separation column having a solid phase and retaining with the solid phase the iron sucrose.

21. The method of claim 20 further comprising removing with an eluant the iron sucrose from the solid phase and collecting an eluate containing iron sucrose.

22. The method of claim 19 further comprising administering an iron sucrose composition to a subject, the obtaining comprising taking a serum sample from the subject at a time interval after the administering, the time interval selected from the group consisting of 0 minutes, 1 minute, 5 minutes, 10, minutes, 15 minutes, 30 minutes, 45 minutes, 1 hour, 2 hours, 3 hours, 4 hours, 6 hours, 8 hours, 12 hours and 24 hours and combinations thereof.

23. The method of claim 22 further comprising obtaining a plurality of serum samples at a plurality of time intervals and measuring the amount of iron sucrose present in each sample.

24. The method of claim 23 further comprising comparing the amount of iron sucrose present in each sample with a second serum sample containing a second iron sucrose composition having a known bioavailability.
